(19) United States
(12) Patent Application Publication
(10) Pub. No.: US 2020/0214927 A1
(43) Pub. Date: Jul. 9, 2020

(54) DEVICE CONFIGURED TO INDUCE VASODILATION OF A SUPERFICIAL VEIN

(71) Applicants: Glenn Clowney, Smyrna, GA (US); Lauren Clowney, Smyrna, GA (US)

(72) Inventors: Glenn Clowney, Smyrna, GA (US); Lauren Clowney, Smyrna, GA (US)

(21) Appl. No.: 16/242,728

(22) Filed: Jan. 8, 2019

(51) Int. Cl. A61H 11/00; A61H 23/02; A61F 7/00

(52) U.S. Cl. CPC A61H 11/00; A61F 2007/0228; A61F 7/007; A61H 23/02

(57) ABSTRACT

Implementations of a device configured to induce vasodilation of a superficial vein and methods of using the device are provided. The device is configured to ease the process of venipuncture by increasing blood flow to a superficial vein. The device is configured to increase local blood flow to a superficial vein by applying heat and/or vibration at, or near, a prospective puncture site. In this way, vasodilation of the superficial vein may be induced. The device comprises a strap, a flexible heating element, at least one vibration element, a power source, and an ON/OFF switch for selectively energizing both the heating element and the at least one vibration element. In some implementations, the device may further comprise a thermal control switch configured to vary the temperature reached by the heating element and/or a vibratory element control switch configured to vary the vibratory intensity of the at least one vibration element.

### TECHNICAL FIELD

This disclosure relates to implementations of a device configured to induce vasodilation of a superficial vein and methods of using the device.

### BACKGROUND

Venipuncture is the process of obtaining intravenous access to a vein for the purpose of blood sampling or intravenous therapy. This procedure is typically performed by phlebotomist, laboratory scientist, and health care providers. During a venipuncture procedure, blood is usually obtained from the superficial veins of an arm, but blood may also be obtained from the superficial veins of a leg, a hand, or the neck.

Each venipuncture attempt damages the vein wall whether the attempt was successful or not. This consideration is of particular importance when a patient with difficult venous access is encountered. Therefore, increasing the success of each venipuncture attempt is important to patient health. Increasing vein diameter (i.e., vasodilation) is an effective way to increase venipuncture success. Vasodilation of superficial veins may be induced through the application of heat and/or vibratory stimulation to the prospective puncture site.

Accordingly, it can be seen that needs exist for the device configured to induce vasodilation of a superficial vein and the methods of using the device disclosed herein. It is to the provision of a device that is configured to address these needs, and others, that the present invention in primarily directed.

### SUMMARY OF THE INVENTION

Implementations of a device configured to induce vasodilation of a superficial vein and methods of using the device are provided. The device is configured to ease the process of venipuncture by increasing blood flow to a vein that is going to be punctured for the purpose of blood sampling or intravenous therapy. The device is configured to increase local blood flow to a vein by applying heat and/or vibration at, or near, the prospective puncture site. In this way, vasodilation of the superficial vein may be induced.

In some implementations, the device may comprise a strap that can be used to position the device over a prospective puncture site, a heating element able to conform to the contour of a surface, at least one vibration element, a power source, and an ON/OFF switch for selectively energizing both the heating element and the at least one vibration element. The device defines an application area comprising the heating element and the at least one vibration element. The strap can be used to secure the device to a portion of a subject's body (e.g., a leg, an arm, a hand) and/or used to form a grip into which a user (e.g., a subject or a phlebotomist) can insert their hand and thereby position the device over a prospective puncture site.

In some implementations, the device may further comprise a thermal control switch configured to vary the temperature reached by the heating element and/or a vibratory element control switch configured to vary the vibratory intensity of the at least one vibration element.

In some implementations, the strap includes a slot configured to receive the power source therein. The slot may be defined by a strip of material, the ends of which are secured (e.g., sewn) to the strap.

The heating element and the at least one vibration element of the device are secured to a first side of the strap, the heating element overlaps the vibration element(s) thereby forming the application area. In some implementations, the portion of the strap to which the heating element and the at least one vibration element are secured is sufficiently thin, and/or has sufficient thermal transfer characteristics, as to allow the efficient transfer of heat from the heating element, and vibration from the at least one vibration element, to the subject.

### BRIEF DESCRIPTION OF THE DRAWINGS

FIG. 1 illustrates an isometric flat view of a device configured to induce vasodilation of a superficial vein according to the principles of the present disclosure.

FIG. 2 illustrates a top view of the device shown in FIG. 1.

FIG. 3 illustrates a bottom view of the device shown in FIG. 1.

FIG. 4 illustrates an isometric exploded view of the device shown in FIG. 1.

FIG. 5 illustrates the device shown in FIG. 1 being worn on the lower leg of a subject.

FIG. 6 illustrates the device shown in FIG. 1 being worn on an arm of a subject.

FIG. 7 illustrates the device shown in FIG. 1 being worn on the hand of a user.

FIG. 8 is a schematic of an example circuit configured to operate the device shown in FIG. 1.

Like reference numerals refer to corresponding parts throughout the several views of the drawings.

### DETAILED DESCRIPTION

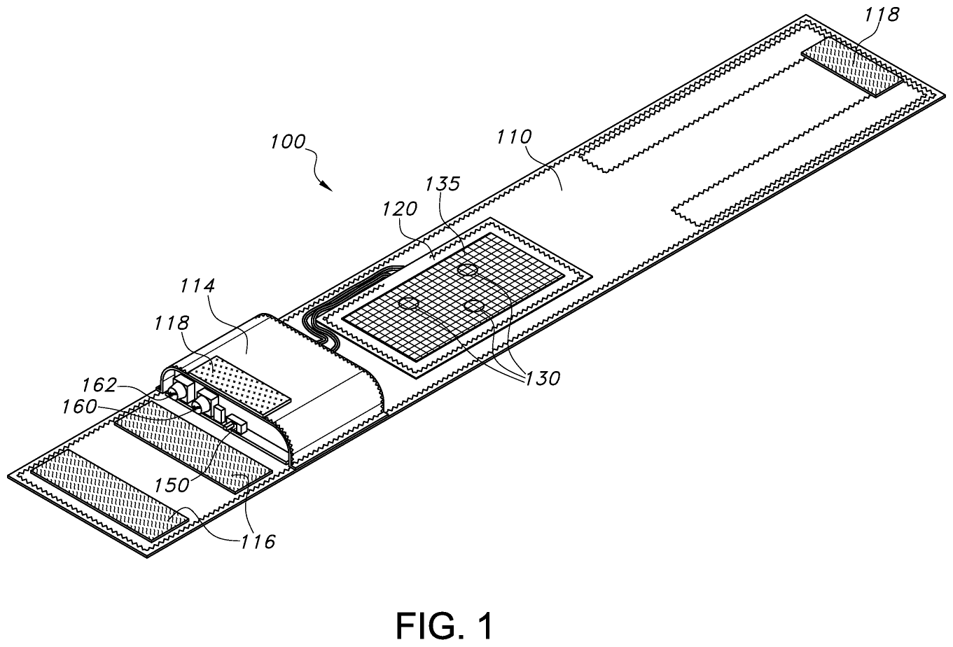

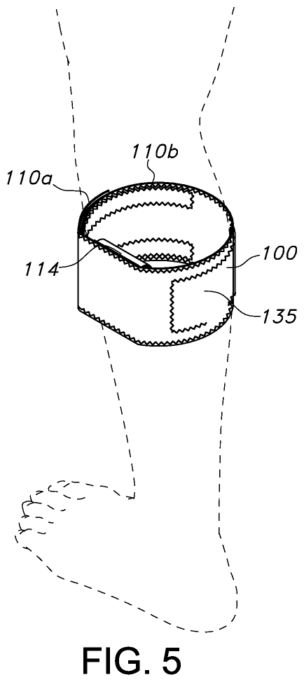

FIGS. 1-4 illustrate an example device 100 configured to induce vasodilation of a superficial vein and FIGS. 5-7 illustrate example methods of using the device 100. The device 100 is configured to ease the process of venipuncture by increasing blood flow to a vein that is going to be punctured for the purpose of blood sampling or intravenous therapy. In some implementations, the device 100 is configured to increase local blood flow to a vein by applying heat and/or vibration at, or near, the prospective puncture site. In this way, vasodilation of the superficial vein may be induced.

As shown in FIGS. 1, 2, and 4, in some implementations, the device 100 may comprise a strap 110, a heating element 120, vibration elements 130, a power source 140, and an ON/OFF switch 150 for selectively energizing both the heating element 120 and the vibration elements 130. The device 100 defines a two-sided application area 135 comprising the heating element 120 and the vibration elements 130. In some implementations, the device 100 may further comprise a thermal control switch 160 and/or a vibratory element control switch 162.

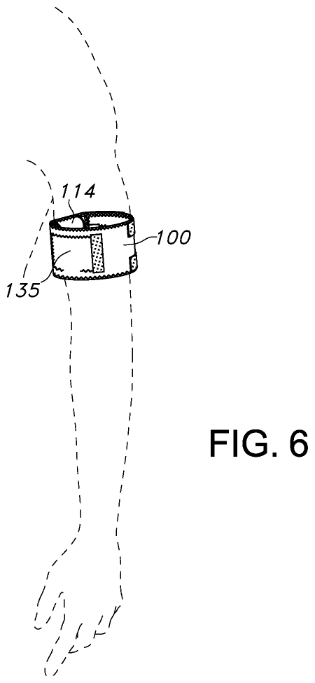

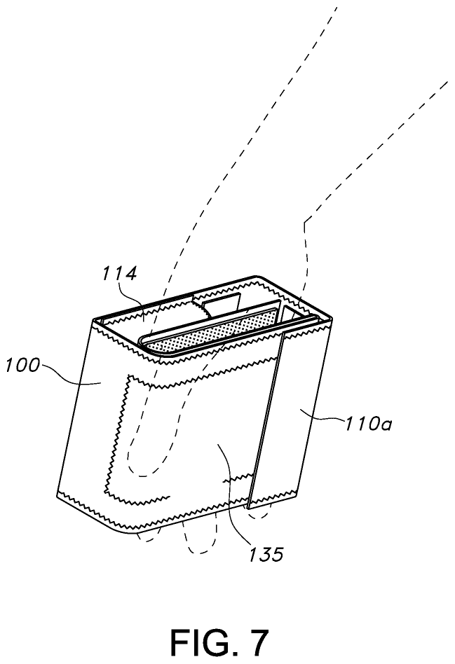

As shown in FIGS. 5-7, the strap 110 can be used to secure the device 100 to a portion of a subject's body (e.g., a leg, an arm, a hand). Prior to using the strap 110 to secure the device 100 to a portion of a subject's body (e.g., a leg or an arm), the application area 135 (i.e., the heating element 120 and the vibration elements 130) should be positioned over a prospective puncture site (see, e.g., FIGS. 5 and 6). Alternatively, the strap 110 could be used to secure the device 100 to a user's hand so that the application area 135 is positioned over the palm thereof (see, e.g., FIG. 7). In this way, the user (e.g., a subject or a phlebotomist) can position and hold the application area 135 of the device 100 over a prospective puncture site. As another alternative, the application area 135 of the device 100 could be positioned over a prospective puncture site on the neck of a subject, the ends of the strap 110 could then be pulled and thereby used to hold the application area 135 of the device 100 in contact with the prospective puncture site (not shown).

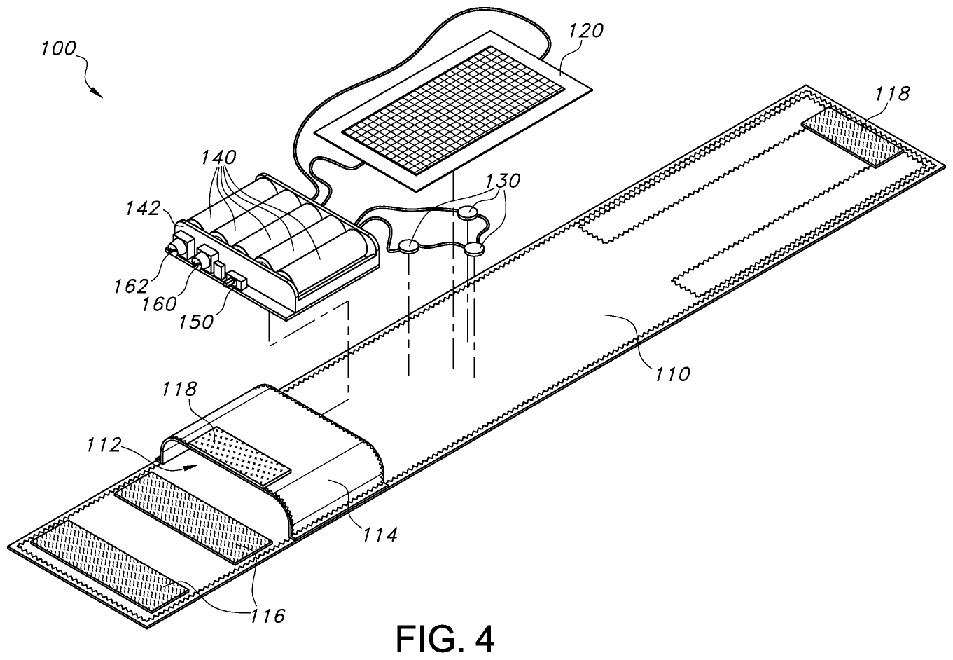

In some implementations, the strap 110 may be a flexible length of material (e.g., one or more layers of neoprene or another suitable material) capable of being wrapped about a portion of a subject's body. The strap 110 includes a slot 112 configured to receive the power source 140 therein (see, e.g., FIG. 4). The slot 112 may be defined by a strip of material 114, the ends of which are secured (e.g., sewn) to the strap 110. The heating element 120 and the vibration elements 130 of the device 100 are secured to a first side of the strap 110, the heating element 120 overlaps the vibration elements 130 thereby forming the application area 135. In some implementations, the portion of the strap 110 to which the heating element 120 and the vibration elements 130 are secured is sufficiently thin, and/or has sufficient thermal transfer characteristics, as to allow the efficient transfer of heat from the heating element 120 and vibration from the vibration elements 130 to the subject. The ends of the strap 110 include hook-and-loop fasteners 116 for securing the device 100 about a portion of a subject's body. In some implementations, the ends of the strap 110 may include another type of fastening device (e.g., a buckle) in-lieu of hook-and-loop fasteners 116. In some implementations, as discussed in greater detail below, the strap 110 may include additional fasteners 118 (e.g., hook-and-loop fasteners) that are used to secure the device 110 about the hand of a user and/or for forming the strap 110 into a grip that can be held (see, e.g., FIG. 7).

In some implementations, the heating element 120 may be a flexible heating pad. By being flexible, the heating element 120 is able to conform to the contour of a surface (e.g., a portion of a subject's body). When the ON/OFF switch 150 is used to close the circuit 171, the heating element 120 heats up. In some implementations, the thermal control switch 160 may be used to vary the voltage applied to the heating element 120. In this way, the temperature reached by the heating element 120 can be adjusted (or set). The thermal control switch 160 can be a rotary potentiometer, or another suitable variable resistor.

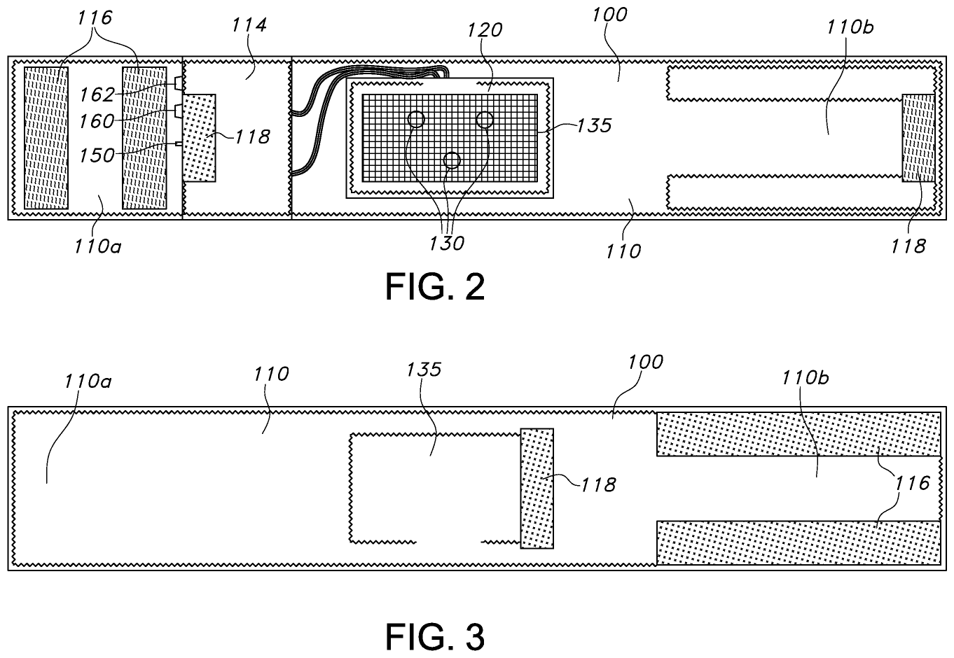

As shown in FIGS. 1 and 2, in some implementations, the heating element 120 can be sewn to the strap 110 of the device 100. In some implementations, the heating element 120 may be secured to the strap 110 by a suitable adhesive. One of ordinary skill in the art, having the benefit of the present disclosure, would know of other suitable ways to secure the heating element 120 to the strap 110 of the device 100 (e.g., the heating element 120 could be positioned within a pocket that includes at least one sidewall that is sufficiently thin, or has sufficient thermal transfer characteristics, as to allow the efficient transfer of heat from the heating element 120 to the subject).

In some implementations, each vibration element 130 may be a shaftless vibratory motor. When the ON/OFF switch 150 is used to close the circuit 171, each vibration element 130 begins to vibrate. In some implementations, the vibratory element control switch 162 may be used to vary the voltage applied to the vibration elements 130. In this way, the vibratory intensity of each vibration element 130 can be adjusted (or set). The vibratory element control switch 162 can be a rotary potentiometer, or another suitable variable resistor.

While three vibration elements 130 are shown in the figures, it should be understood that, in some implementations, a device 100 may comprise less than three or more than three vibration elements 130. In some implementations, each vibration element 130 is secured to the strap 110 by an adhesive backing. One of ordinary skill in the art, having the benefit of the present disclosure, would know of other suitable ways to secure each vibration element 130 to the strap 110 of the device 100.

As shown in FIG. 4, in some implementations, the power source 140 may be one or more electrochemical cells (e.g., AA batteries) held by a battery holder 142 that is conductively connected to the heating element 120 and the vibration elements 130. In some implementations, the ON/OFF switch 150, the optional thermal control switch 160, the optional vibratory element control switch 162, or a combination thereof, may be mounted on the battery holder 142. In some implementations, the power source 140 may be a pouch cell (e.g., a lithium-ion polymer battery) held by a battery holder (not shown).

As shown in FIG. 1, in some implementations, the power source 140 can be held within the slot 112 of the strap 110 by friction. In some implementations, the power source 140 may be held within the slot 112 of the strap 110 by a removable adhesive, one or more fasteners, or a combination thereof.

In some implementations, the device 100 may be configured so that a conventional AC power line can be used to power it's circuit 171 in lieu of batteries (not shown).

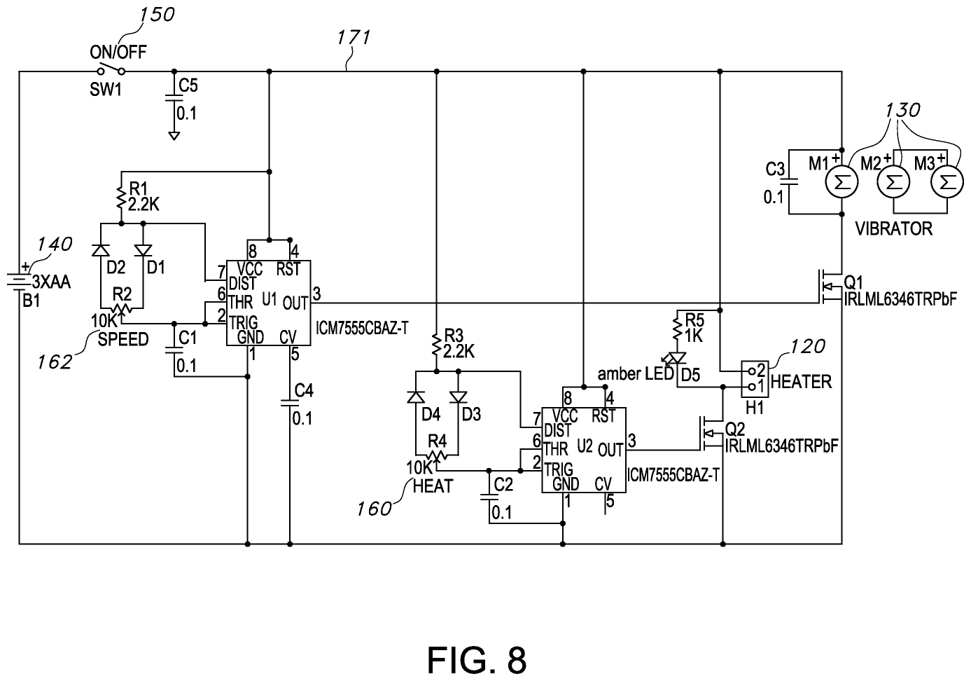

FIG. 8 illustrates an example circuit 171 that is configured to operate the device 100. The example circuit 171 comprises the heating element 120, the vibration elements 130, the power source 140, the ON/OFF switch 150, and in some implementations, the thermal control switch 160 and/or the vibratory element control switch 162. Although not fully disclosed in the drawings, it will be understood that suitable wiring (or traces) connects the electrical components of the device 100 disclosed herein (e.g., the heating element 120, vibration element(s) 130, power source 140, ON/OFF switch 150, and the optional switches 160, 162).

As disclosed above, one or more implementations of the device 100 may be used to induce vasodilation of a superficial vein. In some implementations, the following steps may be taken to induce vasodilation using the device 100 disclosed herein. Initially, the device is turned ON using the provided ON/OFF switch 150. In this way, the heating element 120 and the vibration elements 130 are energized. Then, in some implementations, the temperature and vibratory intensity of the heating element 120 and the vibration elements 130, respectively, may be adjusted (and set) using the provided thermal control switch 160 and vibratory element control switch 162. Next, a side of the application area 135 is positioned over a prospective puncture site. While the application area 135 of the device 100 is positioned on a prospective puncture site and after a sufficient amount of time has passed, any adjacent superficial vein(s) may vasodilate due to the heat and vibratory stimulation emanating from the device 100. Once vasodilation has occurred, the device 100 may be removed and the venipuncture procedure initiated. One of ordinary skill in the art would know how long to leave the device 100 in position for the purpose of inducing vasodilation.

In some implementations, the strap 110 may be used to secure the device 100 to a portion of a subject's body (see, e.g., FIGS. 5 and 6) or used to form a grip into which the user can insert their hand (see, e.g., FIG. 7).

As shown in FIGS. 5 and 6, in some implementations, the following steps may be used to secure the device 100 to a portion of a subject's body (e.g., a leg or an arm). Initially, a first side of the application area 135 is positioned over a prospective puncture site (i.e., the heating element 120 is placed in direct contact with the prospective puncture site). Then, the strap 100 is wrapped about the leg (or arm) and the provided hook-and-loop fasteners 116 are used to secured a first end 110a of the strap 110 to a second end 110b thereof.

As shown in FIG. 7, in some implementations, the following steps may be used so that the device 100 can be easily grasped by a user (e.g., a subject or a phlebotomist). Initially, the second end 110b of the strap 110 is folded over the heating element 120 and secured to the strip of material 114 that defines the slot 112 for the power source 140. The second end 110b of the strap 110 is secured to the strip of material 114 by the provided hook-and-loop fasteners 118. Then, the second end 110b of the strap 110 is folded over again so that the first end 110a of the strap 110 can be secured to the hook-and-loop fasteners 118 located on the second side of the strap 110, adjacent the second side of the application area 135. Next, the user may insert a portion of their hand into an opening formed by the strap 110 and thereby grasp the device 110 prior to applying the second side of the application area 135 to a prospective puncture site.

Reference throughout this specification to “an embodiment” or “implementation” or words of similar import means that a particular described feature, structure, or characteristic is included in at least one embodiment of the present invention. Thus, the phrase “in some implementations” or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

While operations are depicted in the drawings in a particular order, this should not be understood as requiring that such operations be performed in the particular order shown or in sequential order, or that all illustrated operations be performed, to achieve desirable results.

1. A device configured to induce vasodilation of a superficial vein, the device comprising:
a strap that can be used to position the device over a prospective puncture site;
a heating element that is configured to conform to the contour of a surface;
at least one vibration element;
a power source; and
an ON/OFF switch configured to selectively energize the heating element and the at least one vibration element, the ON/OFF switch is conductively connected to the power source, the heating element, and the at least one vibration element;
wherein the device defines an application area comprising the heating element and the at least one vibration element.

2. The device of claim 1, wherein the strap includes a slot configured to receive the power source therein, the slot is defined by a strip of material, each end of the strip of material is secured to the strap.

3. The device of claim 1, wherein the heating element is a flexible heating pad.

4. The device of claim 1, wherein the at least one vibration element is a shaftless vibratory motor.

5. The device of claim 1, wherein the heating element overlaps the at least one vibration element.

6. The device of claim 5, wherein the heating element and the at least one vibration element are secured to a first side of the strap, the portion of the strap to which the heating element and the at least one vibration element are secured is configured to allow heat and vibration generated by the heating element and the at least one vibration element, respectively, to transfer to a second side thereof.

7. A device configured to induce vasodilation of a superficial vein, the device comprising:
a strap that can be used to position the device over a prospective puncture site;
a heating element that is configured to conform to the contour of a surface;
at least one vibration element;
a power source;
an ON/OFF switch configured to selectively energize the heating element and the at least one vibration element, the ON/OFF switch is conductively connected to the power source, the heating element, and the at least one vibration element;
a thermal control switch configured to vary the temperature reached by the heating element, the thermal control switch is conductively connected to the power source and the heating element; and
a vibratory element control switch configured to vary the vibratory intensity of the at least one vibration element, the vibratory element control switch is conductively connected to the power source and the at least one vibration element;
wherein the device defines an application area comprising the heating element and the at least one vibration element.

8. The device of claim 7, wherein the strap includes a slot configured to receive the power source therein, the slot is defined by a strip of material, each end of the strip of material is secured to the strap.

9. The device of claim 7, wherein the heating element is a flexible heating pad.

10. The device of claim 7, wherein the at least one vibration element is a shaftless vibratory motor.

11. The device of claim 7, wherein the heating element overlaps the at least one vibration element.

12. The device of claim 11, wherein the heating element and the at least one vibration element are secured to a first side of the strap, the portion of the strap to which the heating element and the at least one vibration element are secured is configured to allow heat and vibration generated by the heating element and the at least one vibration element, respectively, to transfer to a second side thereof.

13. A method of using a device configured to induce vasodilation of a superficial vein, the method comprising:
providing a device comprising: a strap that can be used to position the device over a prospective puncture site; a heating element that is configured to conform to the contour of a surface; at least one vibration element; a power source; and an ON/OFF switch configured to selectively energize the heating element and the at least one vibration element; wherein the device defines an application area comprising the heating element and the at least one vibration element;
actuating the ON/OFF switch of the device to energize the heating element and the at least on vibration element; and
positioning the application area of the device over a prospective puncture site on a subject.

14. The method of claim 13, further comprising: orienting the application area of the device so that the heating element is placed in direct contact with the prospective puncture site; and wrapping the strap about a limb of the subject and securing a first end of the strap to a second end thereof.

15. The method of claim 13, further comprising: folding the strap to form an opening into which a user may insert a portion of their hand, thereby allowing the user to grasp the device while holding the application area over the prospective puncture site.

16. The method of claim 13, wherein the devices further comprises: a thermal control switch configured to vary the temperature reached by the heating element, and a vibratory element control switch configured to vary the vibratory intensity of the at least one vibration element; the method further comprises:
using the thermal control switch to set the temperature reached by the heating element; and
using the vibratory element control switch to set the vibratory intensity of the at least one vibration element.

17. The method of claim 13, wherein the heating element of the device is a flexible heating pad.

18. The method of claim 13, wherein the at least one vibration element of the device is a shaftless vibratory motor.

19. The method of claim 13, wherein the heating element of the device overlaps the at least one vibration element.

20. The method of claim 19, wherein the heating element and the at least one vibration element of the device are secured to a first side of the strap, the portion of the strap to which the heating element and the at least one vibration element are secured is configured to allow heat and vibration generated by the heating element and the at least one vibration element, respectively, to transfer to a second side thereof.
